(19) United States
(12) Patent Application Publication
(10) Pub. No.: US 2015/0238225 A1
(43) Pub. Date: Aug. 27, 2015

(54) LAPAROSCOPIC SURGICAL APPARATUS

(71) Applicant: SEIKO EPSON CORPORATION, Tokyo (JP)

(72) Inventors: Hirokazu SEKINO, Chino-shi (JP); Hideki KOJIMA, Matsumoto-shi (JP); Koichiro MIYAZAKI, Ina-shi (JP)

(21) Appl. No.: 14/632,877

(22) Filed: Feb. 26, 2015

(30) Foreign Application Priority Data
Feb. 27, 2014 (JP) 2014-036271

(51) Int. Cl. A61B 17/34; A61B 17/00; A61B 18/14; A61B 17/3203

(52) U.S. Cl. CPC A61B 17/3462; A61B 17/3203; A61B 17/00234; A61B 18/1482; A61B 2017/00154; A61B 2017/3464; A61B 2018/00601

(57) ABSTRACT

A fluid ejection device includes a fluid ejection tube having a forward end nozzle inserted into an abdominal cavity. The fluid ejection tube, accommodated in a cylindrical casing section, is movable in an extending direction. The fluid ejection tube moves in the extending direction, interlocked with a sliding operation of a sealing state switching operation section on the outside of the casing section. Fixed and sliding sealing sections, which seal the inside of the abdominal cavity off from outside, are arranged between the fluid ejection tube and an inner wall surface of the casing section. The fixed and sliding sealing sections shift from a first sealing state with a first airtightness to a second sealing state with a higher airtightness, through movement of the fluid ejection tube by the sealing state switching operation section. The fluid ejection device is electrically drivable when the second sealing state is established.

### CROSS-REFERENCE

This application claims the benefit of Japanese Patent Application No. 2014-36271, filed on Feb. 27, 2014. The content of the aforementioned application is incorporated herein by reference in its entirety.

### BACKGROUND

1. Technical Field

The present invention relates to a laparoscopic surgical apparatus.

2. Related Art

In a laparoscopic surgical operation, a plurality of holes approximately 5 to 10 mm is opened in the abdominal part of a patient, and a cylindrical member called a trocar is installed in each of these holes. Surgical instruments used for treatment inside the abdominal cavity, such as laparoscopic camera, surgical knife, and forceps, are introduced into the abdominal cavity via the cylindrical hole of each trocar. JP-A-2000-262528 proposes an instrument having a combination of the functions of an electric surgical knife and a water-jet surgical knife, as a surgical instrument used for laparoscopic surgical operations.

In laparoscopic surgical operations, sealability between the inside of the abdominal cavity and the outside needs to be secured in order to restrain contraction of the abdominal cavity due to a leakage of gas. Therefore, normally, a sealing member for securing sealability to the surgical instrument is arranged on the cylindrical inner wall surface of the trocar. However, there is a demand for enhanced airtightness in the abdominal cavity during laparoscopic surgical operations. There is also a demand for reduction in size and weight of surgical instruments, improvement in user-friendliness, reduction in costs, saving of resources, easier manufacturing, and the like.

### SUMMARY

An advantage of some aspects of the invention is to solve at least a part of the problems described above, and the invention can be implemented as the following forms.

[1] An aspect of the invention provides a laparoscopic surgical apparatus. The laparoscopic surgical apparatus may include a surgical instrument section, an accommodation section, an operation section, and a sealing section. The surgical instrument section may have an extension site having at least a forward end part thereof inserted into an abdominal cavity, and may be driven by electric power supplied from outside. The accommodation section may be a cylindrical member that accommodates the extension site of the surgical instrument section in such a way that the extension site is movable in an extending direction. The operation section may be an operation section for causing the extension site of the surgical instrument section to move in the extending direction from outside the accommodation section. The sealing section may be arranged between the extension site and the accommodation section in order to seal an inside of the abdominal cavity off from outside. The sealing section may be capable of shifting from a first sealing state with a first airtightness to a second sealing state with a higher airtightness than in the first sealing state, through the movement of the extension site by the operation section. The surgical instrument section may be configured in such a way that the surgical instrument section is in an electrically drivable state when the second sealing state is established. According to this laparoscopic surgical apparatus of this configuration, since the sealing section is provided in the laparoscopic surgical apparatus itself, a leakage of gas from inside the abdominal cavity via the laparoscopic surgical apparatus is restrained. Also, since the laparoscopic surgical apparatus is electrically drivable in the state where internal sealability is enhanced, a leakage of gas from inside the abdominal cavity via the laparoscopic surgical apparatus during a surgical operation is restrained securely.

[2] In the laparoscopic surgical apparatus according to the aspect described above, the surgical instrument section may be in another state than the electrically drivable state when the first sealing state is established. According to the laparoscopic surgical apparatus of this configuration, electric driving of the laparoscopic surgical apparatus in the first sealing state with relatively low internal sealability is restrained.

[3] In the laparoscopic surgical apparatus according to the aspect described above, the sealing section may be in the first sealing state when the forward end part of the extension site is accommodated inside the accommodation section, and the sealing section may shift to the second sealing state when the forward end part of the extension site extends from the accommodation section through the movement of the extension site by the operation section. According to the laparoscopic surgical apparatus of this configuration, an operation to an operative field becomes possible when sealability inside the apparatus is enhanced.

[4] In the laparoscopic surgical apparatus according to the aspect described above, the surgical instrument section may include first and second surgical instrument sections, each having an extension site extending in parallel within the accommodation section. The operation section may include first and second operation sections for operating the respective extension sites of the first and second surgical instrument sections. The sealing section may include first and second sealing sections provided between the respective extension sites of the first and second surgical instrument sections and the accommodation section. The first surgical instrument section may be in an electrically drivable state when the first sealing section is in the second sealing state. The second surgical instrument section may be in an electrically drivable state when the second sealing section is in the second sealing state. According to the laparoscopic surgical apparatus of this configuration, sealability inside the laparoscopic surgical apparatus having a combination of the first and second surgical instrument sections is secured.

[5] In the laparoscopic surgical apparatus according to the aspect described above, the operation section may have a slider section that is slidable outside the accommodation section, and a cylindrical holding section that airtightly holds an outer periphery of the surgical instrument section inside the accommodation section and is connected to the slider section. The extension site of the surgical instrument section may move within the accommodation section along with the holding section when the slider section is operated to slide. The sealing section may be arranged to annularly surround an outer periphery of the extension site. The first sealing state of the sealing section may be a state where an inner peripheral part of the sealing section and an outer peripheral part of the holding section contact each other airtightly. The second sealing state may be a state where the holding section is situated deeper into the sealing section than in the first sealing state. According to the laparoscopic surgical apparatus of this configuration, the sealing state of the laparoscopic surgical apparatus can be switched, using a simple configuration.

[6] The laparoscopic surgical apparatus according to the aspect described above may further include a drive control unit that controls electric driving of the surgical instrument section, a first electrode section that moves, interlocked with the slider section, and a second electrode section fixed to the accommodation section. The first and second electrode sections may contact each other when the sealing section moves to a position where the second sealing state is established, through the sliding of the slider section. The drive control unit may perform control in such a way that electric power from outside can be supplied to the surgical instrument section when the first and second electrode sections contact each other. According to the laparoscopic surgical apparatus of this configuration, the control of electric driving of the laparoscopic surgical apparatus can be interlocked with the switching of the sealing state of the laparoscopic surgical apparatus, using a simple configuration.

[7] The laparoscopic surgical apparatus according to the aspect described above may further include a report section that reports that the surgical instrument section is in an electrically drivable state. According to the laparoscopic surgical apparatus of this configuration, convenience for the operator is enhanced by restraining operation errors of the laparoscopic surgical apparatus or the like.

[8] In the laparoscopic surgical apparatus according to the aspect described above, the surgical instrument section may include a fluid ejection section that ejects a fluid to an operative field inside the abdominal cavity from the forward end part. According to the laparoscopic surgical apparatus of this configuration, a leakage of gas from inside the abdominal cavity is restrained when a surgical instrument ejecting a fluid such as a water-jet surgical knife is used.

Not all of the plurality of components provided in each of the aspects of the invention is essential. A part of the plurality of components can be changed, deleted, replaced with another new component, or partly deleted in terms of its limiting content, in order to solve a part or all of the foregoing problems or in order to achieve apart of or the entire advantageous effects described in this description. Also, a part or all of the technical features included in one of the aspects of the invention can be combined with a part or all of the technical features included in another aspect of the invention to form one independent aspect of the invention, in order to solve a part or all of the foregoing problems or in order to achieve a part or all of the advantageous effects described in this description.

The invention can be implemented in various other forms than the laparoscopic surgical apparatus. For example, the invention can be implemented in the form of a surgical system having a laparoscopic surgical apparatus, a method for using or controlling a laparoscopic surgical apparatus, a computer program that realizes these methods, a non-temporary recording medium on which the computer program is recorded, and the like.

### BRIEF DESCRIPTION OF THE DRAWINGS

The invention will be described with reference to the accompanying drawings, wherein like numbers reference like elements.

FIG. 1 is a schematic view showing the configuration of a fluid ejection device according to a first embodiment.

FIGS. 2A to 2C are explanatory views illustrating the switching of the state of the fluid ejection device.

FIG. 3 is a schematic view showing the configuration of a combination surgical device according to a second embodiment.

FIGS. 4A and 4B are schematic views illustrating the internal configuration of an exterior section of a handpiece.

FIGS. 5A and 5B are explanatory views illustrating the switching of the state of the combination surgical device.

FIGS. 6A and 6B are explanatory views illustrating the switching of the state of the combination surgical device.

### DESCRIPTION OF EXEMPLARY EMBODIMENTS

### A. First Embodiment

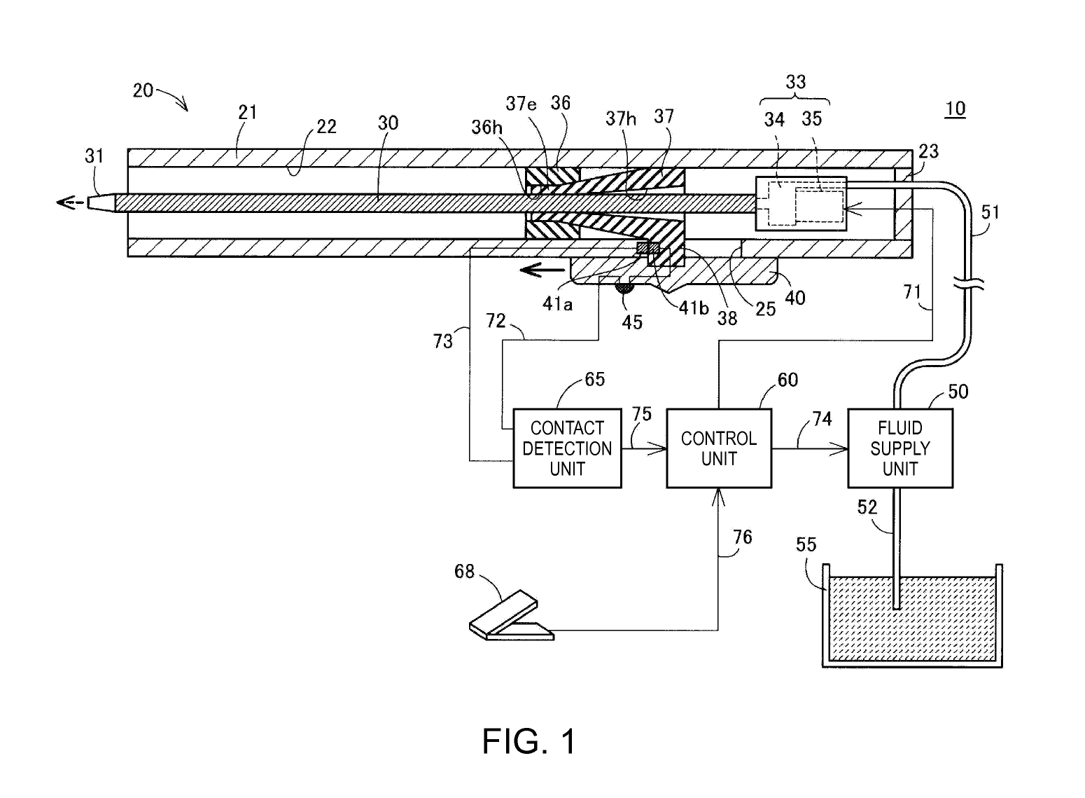

FIG. 1 is a schematic view showing the configuration of a fluid ejection device 10 that is a laparoscopic surgical apparatus as a first embodiment of the invention. The fluid ejection device 10 functions as a water-jet surgical knife which ejects a fluid provided with pulsation (pulsating flow) and thus performs incision or excision or the like on an affected part. The fluid ejection device 10 has a handpiece 20, a fluid supply unit 50, a fluid storage unit 55, a control unit 60, a contact detection unit 65, and a foot switch 68.

The handpiece 20 is a stick-like surgical instrument section which an operator as a user holds in a hand to operate. In a laparoscopic surgical operation, the operator holds the rear end side (right side on the sheet of FIG. 1) of the handpiece 20 and inserts the forward end side (left side on the sheet of FIG. 1) of the handpiece 20 into the abdominal cavity via the cylindrical hole of a trocar installed on the abdominal part of the patient. The handpiece 20 has a casing section 21, a fluid ejection tube 30, an ejection drive section 33, a fixed sealing section 36, a sliding sealing section 37, a sealing state switching operation section 40, first and second electrode sections 41a, 41b, and a report section 45.

The casing section 21 is a cylindrical accommodation section and accommodates the fluid ejection tube 30, the ejection drive section 33, the fixed sealing section 36 and the sliding sealing section 37, inside a cylindrical hole 22. The forward end side of the casing section 21 is opened so that a nozzle 31 of the fluid ejection tube 30 can extend. The rear end side of the casing section 21 is closed by having a cap section 23 attached thereon. In the cap section 23, a penetration hole is provided so that a supply tube 51 and a signal line 71 can be introduced into the casing section 21.

The fluid ejection tube 30 and the ejection drive section 33 are equivalent to a surgical instrument section of the fluid ejection device 10. The fluid ejection tube 30 is a tubular extension site extending in the cylindrical hole 22 of the casing section 21. The nozzle 31 as an opening section for ejecting a fluid is provided at the forward end part of the fluid ejection tube 30. The rear end part of the fluid ejection tube 30 is connected to a fluid accommodation chamber 34 of the ejection drive section 33. The fluid ejection tube 30 and the ejection drive section 33 are displaced in the extending direction of the fluid ejection tube 30 in the casing section 21 according to the operation of the sealing state switching operation section 40 by the operator.

The ejection drive section 33 has the fluid accommodation chamber 34 and a pulsation generation section 35. The fluid accommodation chamber 34 is filled with a fluid supplied from the fluid supply unit 50 via the supply tube 51. The pulsation generation section 35 has a piezoelectric element and a diaphragm (not shown). The piezoelectric element of the pulsation generation section 35 expands in response to a drive signal transmitted from the control unit 60 via the signal line 71 and causes the diaphragm forming a wall section of the fluid accommodation chamber 34 to flex and deform. Thus, the volume of the fluid accommodation chamber 34 changes and a pulsating flow flows into the fluid ejection tube 30 from the fluid accommodation chamber 34. The pulsating flow flowing in the fluid ejection tube 30 is ejected from the nozzle 31. The ejection drive section 33 may not be accommodated in the cylindrical hole 22 and may be arranged outside the casing section 21. In such a case, the fluid ejection tube 30 is inserted through the penetration hole of the cap section 23 and connected to the ejection drive section 33 arranged outside the casing 21.

The fixed sealing section 36 and the sliding sealing section 37 tightly contact each other in a fitting manner and thus seals the inside of the abdominal cavity off from the outside. The fixed sealing section 36 is made up of a cylindrical sealing member. The outer peripheral surface of the fixed sealing section 36 is airtightly fixed to the inner peripheral wall surface of the casing section 21. The fluid ejection tube 30 is inserted through a cylindrical hole 36h in the center of the fixed sealing section 36. The inner diameter of the cylindrical hole 36h of the fixed sealing section 36 is formed to be greater than the outer diameter of the fluid ejection tube 30 so that a gap for a forward end part 37e of the sliding sealing section 37 to enter into is formed between the fixed sealing section 36 and the fluid ejection tube 30.

The sliding sealing section 37 is arranged further toward the rear end side than the fixed sealing section 36, within the casing section 21. The sliding sealing section 37 is made up of a cylindrical sealing member having an outer diameter like a substantially conical shape which decreases in diameter in a tapered shape from the rear end toward the forward end. The fluid ejection tube 30 is inserted through a cylindrical hole 37h of the sliding sealing section 37. The cylindrical hole 37h of the sliding sealing section 37 decreases in diameter toward the forward end. The sliding sealing section 37 holds the fluid ejection tube 30 tightly and airtightly at the forward end part 37e thereof. That is, the sliding sealing section 37 also functions as a holding section for the fluid ejection tube 30.

The sliding sealing section 37 is slidable in the extending direction of the fluid ejection tube 30 within the cylindrical hole 22 of the casing section 21 in the state where the outer peripheral surface on the rear end side airtightly contacts the inner wall surface of the casing section 21. Since the fluid ejection tube 30 is held by the forward end part 37e of the sliding sealing section 37 as described above, the fluid ejection tube 30 moves in the extending direction thereof with the slide of the sliding sealing section 37. When the sliding sealing section 37 moves to the forward end side and the forward end part 37e thereof enters into the cylindrical hole 36h of the fixed sealing section 36, the space on the forward end side from the fixed sealing section 36 and the space on the rear end side from the sliding sealing section 37 are sealed with respect to each other.

Sealability between the fixed sealing section 36 and the sliding sealing section 37 changes depending on the depth at which the forward end part 37e of the sliding sealing section 37 enters into the cylindrical hole 36h of the fixed sealing section 36. As described below, the fluid ejection device 10 of this embodiment switches between a “first sealing state” where sealability between the fixed sealing section 36 and the sliding sealing section 37 is low and a “second sealing state” where sealability is higher than in the “first sealing state”.

A protrusion 38 protruding in the direction of diameter is provided on the rear end side of the sliding sealing section 37. The protrusion 38 protrudes outside the casing section 21 via a substantially rectangular penetration window 25 provided on the lateral side of the casing section 21. The protrusion 38 is connected to the sealing state switching operation section 40 on the outside of the casing section 21.

The sealing state switching operation section 40 is made up of a substantially rectangular parallelepiped member. The sealing state switching operation section 40 is arranged on the lateral side of the casing section 21 in a way of closing the penetration window 25 of the casing section 21. The protrusion 38 of the sliding sealing section 37 is fitted in a middle part of the surface facing the casing section 21 of the sealing state switching operation section 40.

The sealing state switching operation section 40 is equivalent to a slider section and is configured to be slidable in the extending direction along the lateral side of the casing section 21. The operator can displace the sealing state switching operation section 40 by pressing the sealing state switching operation section 40 with a finger. Since the sealing state switching operation section 40 is connected to the protrusion 38 of the sliding sealing section 37 as described above, the sliding sealing section 37 is displaced according to the displacement of the sealing state switching operation section 40. Thus, the sealing state between the fixed sealing section 36 and the sliding sealing section 37 changes.

The first and second electrode sections 41a, 41b are arranged in such a way as to be able to contact each other and electrically connect to each other when the sealing state switching operation section 40 moves to the furthest forward end side. In this embodiment, the first electrode section 41a is buried in an inner wall surface facing the rear end side of the penetration window 25 of the casing section 21. The second electrode section 41b is buried in a wall surface facing the forward end side of the protrusion 38 of the sliding sealing section 37. The first and second electrode sections 41a, 41b are connected to the contact detection unit 65 via signal lines 72, 73, respectively. The contact detection unit 65 applies a voltage to the signal lines 72, 73 and thereby detects whether the first and second electrode sections 41a, 41b are electrically connected to each other or not.

The report section 45 is arranged on the surface of the sealing state switching operation section 40 and is made up of a light emitting diode, for example. The report section 45 is connected to the signal line 72, and emits light with a current flowing between the signal lines 72, 73 when the first and second electrode sections 41a, 41b contact each other. Thus, the operator can learn that the first and second electrode sections 41a, 41b are electrically connected.

The fluid supply unit 50 has a pump (not shown). As the pump is driven, the fluid supply unit 50 sucks a fluid (in this embodiment, a physiological saline solution) stored in the fluid storage unit 55 via a resupply tube 52 and supplies the fluid to the fluid accommodation chamber 34 of the ejection drive section 33 via the supply tube 51. The fluid supply unit 50 is connected to the control unit 60 via a signal line 74 and starts supplying the fluid to the ejection drive section 33 in response to a command from the control unit 60.

The control unit 60 is made up of a microcomputer having a main storage device and a central processing unit. The control unit 60 controls the supply of the fluid by the fluid supply unit 50 and also controls the driving of the ejection drive section 33. The control unit 60 is connected to the contact detection unit 65 via a signal line 75 and connected to the foot switch 68 via a signal line 76. The foot switch 68 is a drive switch which the operator can press and operate with a foot.

When the electrically connected state of the first and second electrode sections 41a, 41b is detected by the contact detection unit 65, the control unit 60 transmits a drive signal to the ejection drive section 33 and the fluid supply unit 50 as the operator steps on the foot switch 68 to turn on the foot switch 68. This starts the ejection of a pulsating flow from the nozzle 31.

On the other hand, when the electrically connected state of the first and second electrode sections 41a, 41b is not detected by the contact detection unit 65, the control unit 60 does not transmit a drive signal to the ejection drive section 33 and the fluid supply unit 50 even if the operator steps on the foot switch 68. Therefore, unless the first and second electrode sections 41a, 41b are electrically connected, no pulsating flow is ejected from the nozzle 31. In this way, in the fluid ejection device 10 of this embodiment, the drive control of the fluid ejection device 10 interlocked with the switching of the sealing state is realized, using such a simple configuration as the first and second electrode sections 41a, 41b.

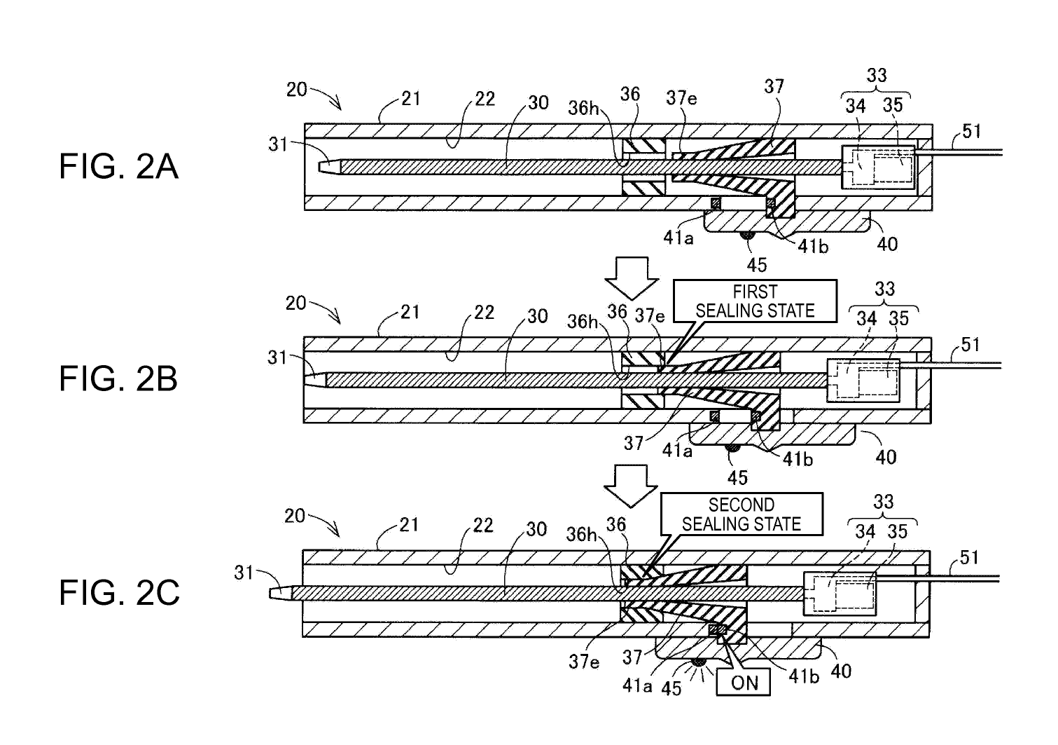

FIGS. 2A to 2C are explanatory views for explaining the switching of the state of the fluid ejection device 10. FIGS. 2A to 2C show different states of the fluid ejection device 10 switched by the operation of the sealing state switching operation section 40. In FIGS. 2A to 2C, the illustration of the other components of the fluid ejection device 10 than the handpiece 20 is omitted as a matter of convenience.

FIG. 2A shows the state when the sealing state switching operation section 40 is at the position on the furthest rear end side within the slidable range. This is the state where the fluid ejection device 10 is not in use and where the fixed sealing section 36 and the sliding sealing section 37 are spaced apart from each other, with no seal formed between the fixed sealing section 36 and the sliding sealing section 37. In this state, the forward end part 37e of the sliding sealing section 37 is opened and the holding of the fluid ejection tube 30 by the sliding sealing section 37 is lowered. Therefore, the fluid ejection tube 30 can be replaced easily.

Also, in this state, since the first and second electrode sections 41a, 41b do not contact each other, driving of the fluid ejection device 10 due to an operation error is restrained. Moreover, in this state, the nozzle 31 is accommodated within the cylinder of the casing section 21. Therefore, even if the handpiece 20 is dropped by mistake, damage to the nozzle 31 is restrained.

FIG. 2B shows the state of the fluid ejection device 10 when the sealing state switching operation section 40 is at the position in the middle of the slidable range. This is the “first sealing state”, where the forward end part 37e of the sliding sealing section 37 is inserted in the cylindrical hole 36h of the fixed sealing section 36, establishing sealability between the fixed sealing section 36 and the sliding sealing section 37. Therefore, in this state, the forward end part of the fluid ejection device 10 can be inserted into the abdominal cavity.

Also, in this state, since the nozzle 31 is accommodated and projected in the casing section 21, damage to the nozzle 31 is restrained when the nozzle 31 is inserted into the abdominal cavity. Moreover, in this state, since the first and second electrode sections 41a, 41b are in a non-contact state, the fluid ejection device 10 is not in an electrically drivable state. Therefore, ejection of a pulsating flow before the arrival of the nozzle 31 at the operative field due to an operation error is restrained.

FIG. 2C shows the state when the sealing state switching operation section 40 is at the position on the furthest forward end side within the slidable range. This state is the “second sealing state”, where the forward end part 37e of the sliding sealing section 37 is inserted deeper into the cylindrical hole 36h of the fixed sealing section 36 than in the first sealing state, thus achieving higher sealability than in the first sealing state.

In this state, the first and second electrode sections 41a, 41b are electrically connected and the fluid ejection device 10 is in an electrically drivable state. Also, the nozzle 31 protrudes from the forward end of the casing section 21. In this state, the operator can cause a pulsating flow to be ejected from the nozzle 31 by stepping on the foot switch 68. Also, the operator can confirm that the fluid ejection device 10 is in an electrically drivable state, via the light emission at the report section 45.

As described above, according to the fluid ejection device 10 of this embodiment, a leakage of gas from inside the abdominal cavity via the cylindrical hole 22 of the handpiece 20 during a surgical operation is restrained by the fixed sealing section 36 and the sliding sealing section 37 within the handpiece 20. Also, according to the fluid ejection device 10 of this embodiment, since ejection of a pulsating flow becomes possible when the device shifts to the second sealing state with higher sealability, driving of the fluid ejection device 10 in a low-sealability state is restrained. Moreover, switching between the sealing states can be achieved easily by the sliding operation of the sealing state switching operation section 40.

### B. Second Embodiment

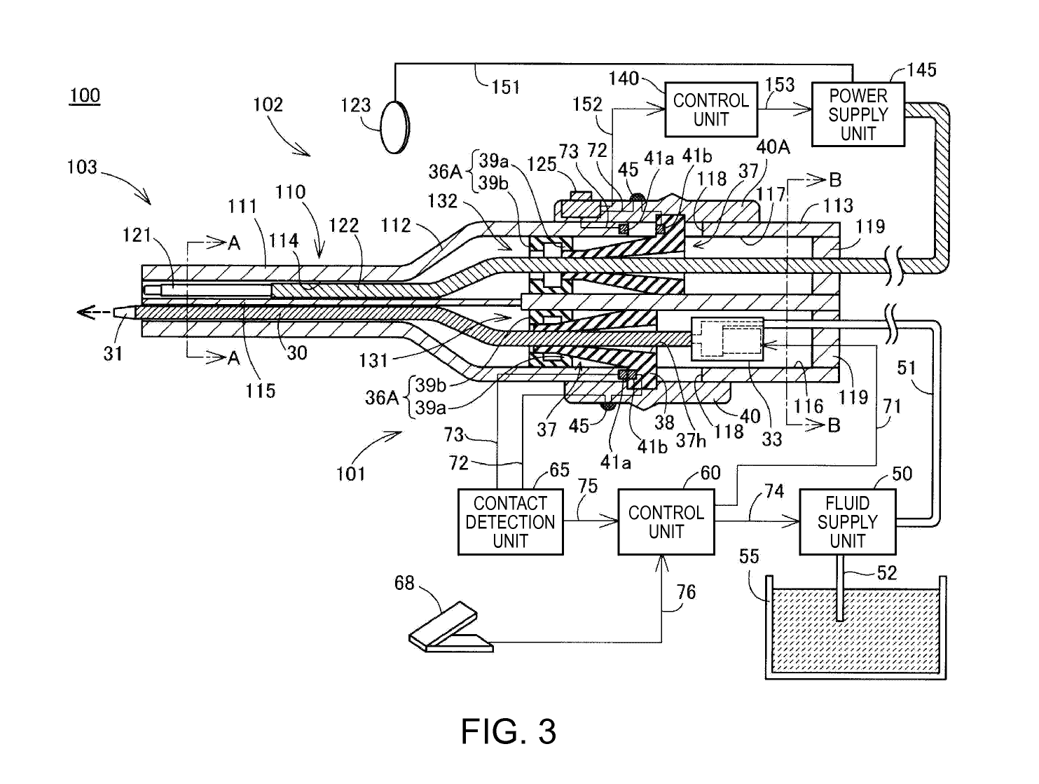

FIG. 3 is a schematic view showing the configuration of a combination surgical device 100 that is a laparoscopic surgical apparatus as a second embodiment of the invention. In FIG. 3, the same components as described in the first embodiment are denoted by the same reference numerals. The combination surgical device 100 has a fluid ejection section 101 that functions as a water-jet surgical knife, and a current application section 102 that functions as a monopolar electric surgical knife. The combination surgical device 100 is configured in such way that a single handpiece 103 can switch the two functions for use.

The fluid ejection section 101 is a first surgical instrument section and has the same configuration part as the fluid ejection device 10 of the first embodiment. The fluid ejection section 101 ejects a pulsating flow, using similar control and mechanism to those of the fluid ejection device 10 of the first embodiment. The explanation of the detailed configuration of the fluid ejection section 101 is omitted as a matter of convenience.

The current application section 102 is a second surgical instrument section and has a forward end surgical knife section 121, a cable section 122, a return electrode 123, a drive switch section 125, a control unit 140, and a power supply unit 145. The forward end surgical knife section 121 is a site extending in the form of a stick. At the time of a surgical operation, the forward end surgical knife section 121 is inserted into the abdominal cavity, applies a high-frequency current to the operative field from the forward end thereof, and thus performs excision, incision or the like on the affected part.

The cable section 122 is formed by a flexible conductive wire for high-frequency current. The cable section 122 is connected to the rear end side of the forward end surgical knife section 121. The forward end surgical knife section 121 is supplied with a high-frequency current from the power supply unit 145 via the cable section 122. The return electrode 123 is a plate-like electrode member attached to the patient at the time of a surgical operation, and functions as the counter electrode of the forward end surgical knife section 121. The return electrode 123 is connected to the power supply unit 145 via a conductive cable 151.

The drive switch section 125 is a button-type switch which the operator can press with a finger. The drive switch section 125 is connected to the control unit 140 via a signal line 152 and transmits a signal indicating a button operation by the operator to the control unit 140. However, as described below, the drive switch section 125 does not transmit a signal indicating a button operation by the operator to the control unit 140 when a second sealing section 132 in the handpiece 103 is not in the second sealing state. In the second embodiment, the drive switch section 125 is integrated with a second sealing state switching operation section 40A.

The control unit 140 is made up of a microcomputer having a central processing unit and a main storage device, and controls driving of the current application section 102. When the control unit 140 receives a signal indicating that the operator is pressing the button from the drive switch section 125, the control unit 140 gives a command to the power supply unit 145 to start supplying a high-frequency current via a signal line 153. The power supply unit 145 outputs a high-frequency current to the cable section 122 in response to the command from the control unit 140. The control unit 140 may be configured integrally with the control unit 60 of the fluid ejection section 101.

The handpiece 103 has an exterior section 110, first and second sealing sections 131, 132, and first and second sealing state switching operation sections 40, 40A. The exterior section 110 has a forward end site 111, a tapered site 112, and a rear end site 113. The forward end site 111 is a cylindrical site to be inserted into the abdominal cavity via a trocar and has a relatively small diameter. The rear end site 113 is a site to be held by the operator and has a relatively large diameter. The tapered site 112 is a follow site with the diameter thereof decreasing toward the forward end, between the forward end site 111 and the rear end site 113.

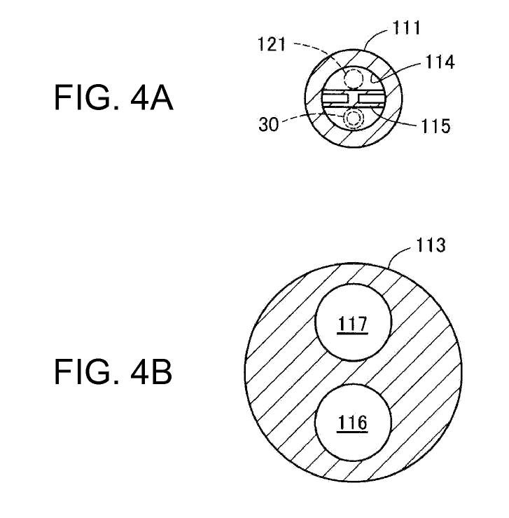

FIGS. 4A and 4B are schematic views for explaining the internal configuration of the exterior section 110 of the handpiece 103. FIGS. 4A and 4B show schematic cross-sections of the exterior section 110 taken along A-A and B-B in FIG. 3, respectively. A cylindrical hole 114 of the forward end site 111 of the exterior section 110 is divided into sections by a guide plate 115 (FIG. 4A). The guide plate 115 extends from the end surface of the rear end site 113 toward the forward end, in the center of the cylindrical hole 114, and guides the movement of the fluid ejection tube 30 of the fluid ejection section 101 and of the forward end surgical knife section 121 and the cable section 122 of the current application section 102. In the rear end site 113 of the exterior section 110, first and second penetration holes 116, 117 for accommodating each component of the fluid ejection section 101 and the current application section 102 are provided in parallel (FIG. 4B).

In the first penetration hole 116 in the rear end site 113, the fluid ejection tube 30 of the fluid ejection section 101 is inserted and the ejection drive section 33 is accommodated (FIG. 3). Meanwhile, in the second penetration hole 117, the cable section 122 of the current application section 102 is inserted. The first and second penetration holes 116, 117 join together in the cylindrical hole 114 of the forward end site 111 and the internal space of the tapered site 112. The fluid ejection tube 30 and the cable section 122 extending from the first and second penetration holes 116, 117 come close to each other in the tapered site 112 and are inserted in the cylindrical hole 114 of the forward end site 111. The openings on the rear end side of the first and second penetration holes 116, 117 are closed by a cap section 119.

The first and second sealing sections 131, 132 are arranged short of the tapered site 112 in the first and second penetration holes 116, 117, respectively. The first and second sealing sections 131, 132 each have a fixed sealing section 36A and a sliding sealing section 37. The fixed sealing section 36A has substantially the same configuration as the fixed sealing section 36 of the first embodiment, except that the fixed sealing section 36A has two circular sealing wall sections 39a, 39b with the same diameter. The first sealing wall section 39a is arranged on the rear end side. The second sealing wall section 39b is arranged on the forward end side.

The sliding sealing section 37 has the same configuration as described in the first embodiment. In the cylindrical hole 37h of each sliding sealing section 37, the fluid ejection tube 30 of the fluid ejection section 101 or the cable section 122 of the current application section 102 is inserted and held. As the sliding sealing section 37 moves toward the forward end side, the forward end part 37e thereof fits sequentially in the openings of the first and second sealing wall sections 39a, 39b of the fixed sealing section 36A.

In the first and second sealing sections 131, 132, the state where the forward end part 37e of the sliding sealing section 37 fits in the opening of the first sealing wall section 39a of the fixed sealing section 36A is called a “first sealing state”. Meanwhile, the state where the forward end part of the sliding sealing section 37 fits in the respective openings of the first and second sealing wall sections 39a, 39b of the fixed sealing section 36A is called a “second sealing state”. In the second sealing state, sealability is higher than in the first sealing state.

The protrusion 38 on each sliding sealing section protrudes outside the exterior section 110 via a substantially rectangular penetration window 118 provided on the lateral side of the exterior section 110. The protrusion 38 is connected to the first or second sealing state switching operation section 40, 40A on the outside of the exterior section 110.

The first sealing state switching operation section 40 has substantially the same configuration as the sealing state switching operation section 40 described in the first embodiment. The first sealing state switching operation section 40 is provided on the lateral side of the rear end site 113 of the handpiece 103 and is connected to the sliding sealing section 37 of the first sealing section 131. The operator can move the sliding sealing section 37 of the first sealing section 131 by pressing and sliding the first sealing state switching operation section 40 with a finger. With the movement of the sliding sealing section 37, the fluid ejection tube 30 moves in the extending direction and the sealing state of the first sealing section 131 shifts.

The second sealing state switching operation section 40A has substantially the same configuration as the first sealing state switching operation section 40, except that the second sealing state switching operation section 40A is integrated with the drive switch section 125. The second sealing state switching operation section 40A is connected to the sliding sealing section 37 of the second sealing section 132, on the lateral side of the rear end site 113 of the handpiece 103. The operator can move the sliding sealing section 37 of the second sealing section 132 by pressing and sliding the second sealing state switching operation section 40A with a finger. With the movement of the sliding sealing section 37, the forward end surgical knife section 121 moves in the extending direction and the sealing state of the second sealing section 132 shifts.

In the combination surgical device 100 of the second embodiment, first and second electrode sections 41a, 41b are provided, as in the fluid ejection device 10 of the first embodiment. In the combination surgical device 100 of the second embodiment, the first and second electrode sections 41a, 41b are provided, corresponding to the first and second sealing state switching operation sections 40, 40A, respectively. The electric driving of the fluid ejection section 101 and the current application section 102 is controlled according to the contact state of the corresponding first and second electrode sections 41a, 41b. The electric driving of the fluid ejection section 101 is controlled according to the detection of the contact state of the first and second electrode sections 41a, 41b by the contact detection unit 65, as described in the first embodiment.

Meanwhile, the electric driving of the current application section 102 is controlled in the following manner. The first and second electrode sections 41a, 41b provided corresponding to the current application section 102 are connected to the drive switch section 125 via signal lines 72, 73. The drive switch section 125 is driven by the electrical connection when the first and second electrode sections 41a, 41b contact each other, and the drive switch section 125 transmits a signal indicating a button operation by the operator to the control unit 140. Meanwhile, when the first and second electrode sections 41a, 41b are in a non-contact state, no current flows through the drive switch section 125. Therefore, in this state, no signal is transmitted to the control unit 140 even if the operator presses the button of the drive switch section 125.

A report section 45 similar to the report section described in the first embodiment is provided on each of the first and second sealing state switching operation sections 40, 40A. The report section 45 provided on the first sealing state switching operation section 40 reports to the operator that the fluid ejection section 101 is in an electrically drivable state, by emitting light. The report section 45 provided on the second sealing state switching operation section 40A reports to the operator that the current application section 102 is in an electrically drivable state, by emitting light.

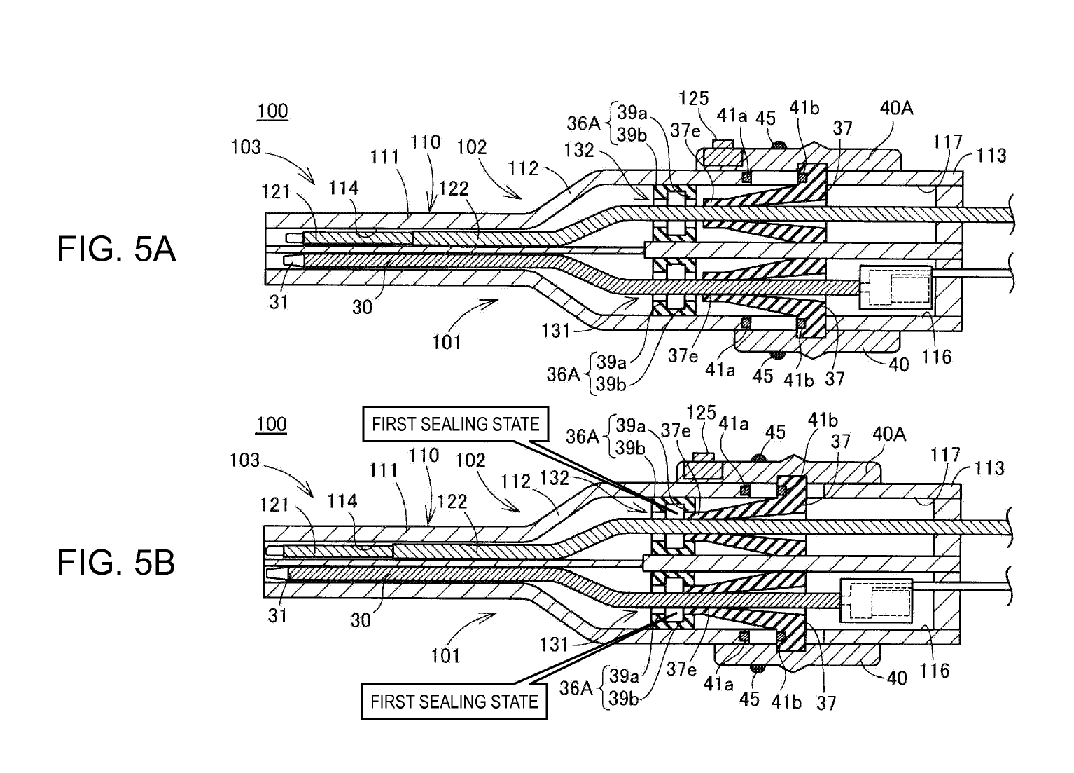

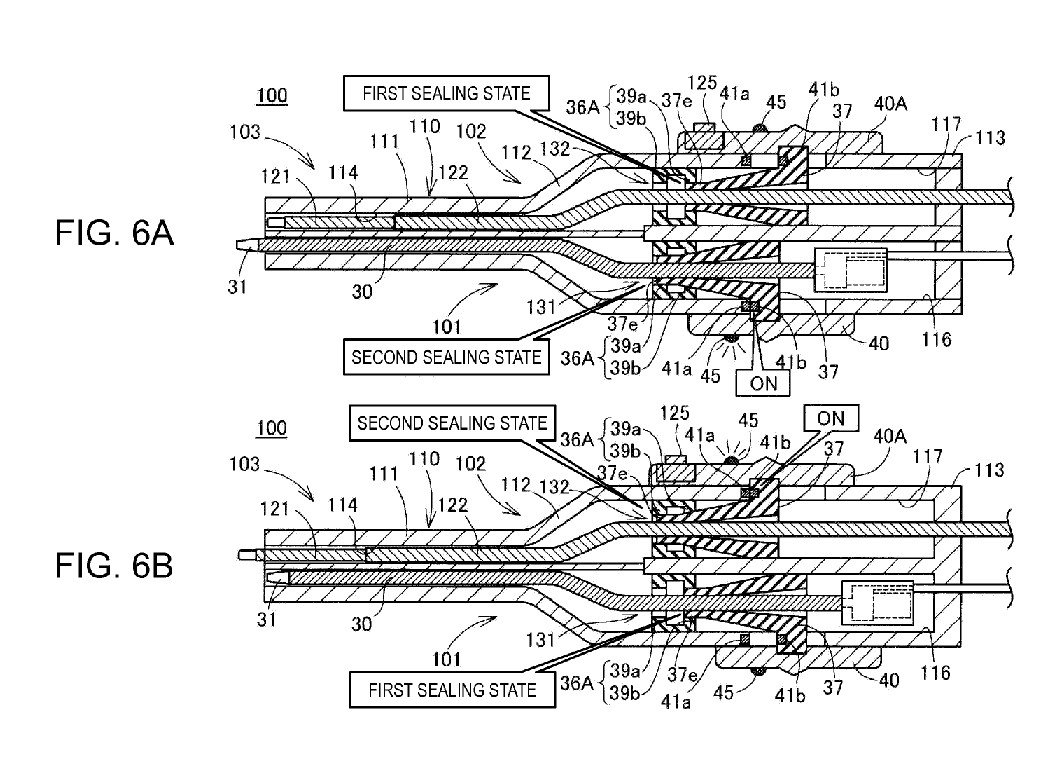

FIGS. 5A and 5B and FIGS. 6A and 6B are explanatory views for explaining the switching of the state of the combination surgical device 100. FIGS. 5A and 5B and FIGS. 6A and 6B show the states of the combination surgical device 100 shifted by the operation of the first and second sealing state switching operation sections 40, 40A. In FIGS. 5A and 5B and FIGS. 6A and 6B, the illustration of the other components of the combination surgical device 100 than the handpiece 103 is omitted as a matter of convenience.

FIG. 5A shows the state where both of the first and second sealing state switching operation sections 40, 40A are at the position on the furthest rear end side within the slidable range. In this state, the fixed sealing section 36A and the sliding sealing section 37 are spaced apart from each other, with no seal formed between the fixed sealing section 36A and the sliding sealing section 37. In this state, since the forward end part 37e of the sliding sealing section 37 is opened, the holding of the fluid ejection tube 30 and the cable section 122 is lowered. Therefore, the fluid ejection tube 30, the forward end surgical knife section 121 and the cable section 122 can be replaced easily.

Also, in this state, since the first and second electrode sections 41a, 41b do not contact each other in either one of the fluid ejection section 101 and the current application section 102, electric driving due to an operation error is restrained. Moreover, in this state, the nozzle 31 of the fluid ejection tube 30 and the forward end part of the forward end surgical knife section 121 are accommodated within the exterior section 110 of the handpiece 103. Therefore, damage to these sections is restrained even if the handpiece 103 is dropped by mistake.

FIG. 5B shows the state where both of the first and second sealing state switching operation sections 40, 40A are at the position in the middle of the slidable range. In this state, since both of the first and second sealing sections 131, 132 are in the first sealing state, the forward end site 111 of the handpiece 103 can be inserted into the abdominal cavity. In this state, since sealability inside the handpiece 103 is established, the forward end site 111 of the handpiece 103 can be inserted into the abdominal cavity.

Also, in this state, since the first and second electrode sections 41a, 41b are in a non-contact state, electric driving of the fluid ejection section 101 and the current application section 102 due to an operation error before the arrival of the forward end of the handpiece 103 at the operative field is restrained. Moreover, the nozzle 31 of the fluid ejection section 101 and the forward end surgical knife section 121 of the current application section 102 are accommodated and protected in the exterior section 110.

FIG. 6A shows the state where the first sealing state switching operation section 40 is at the position on the furthest forward end side within the slidable range, whereas the second sealing state switching operation section 40A remains at the position in the middle of the slidable range. In this state, the first sealing section 131 is in the second sealing state, enhancing sealability in the handpiece 103. Also, the nozzle 31 protrudes from the opening of the exterior section 110, and the fluid ejection section 101 is in an electrically drivable state.

In this state, the operator can cause a pulsating flow to be ejected toward the operative field from the nozzle 31, by stepping on the foot switch 68 (FIG. 3). Meanwhile, since the current application section 102 is in an electrically non-drivable state, driving of the current application section 102 due to an operation error by the operator is restrained. Also, the operator can learn that only the fluid ejection section 101 is in an electrically drivable state, since only the report section 45 in the first sealing state switching operation section 40 emits light.

FIG. 6B shows the state where the second sealing state switching operation section 40A is at the position on the furthest forward end side within the slidable range, whereas the first sealing state switching operation section 40 remains at the position in the middle of the slidable range. In this state, the second sealing section 132 is in the second sealing state, enhancing sealability in the handpiece 103. Also, the forward end part of the forward end surgical knife section 121 protrudes from the opening of the exterior section 110, and the current application section 102 is in an electrically drivable state.

In this state, the operator can cause a high-frequency current to be applied to the operative field via the forward end surgical knife section 121, by pressing the button on the drive switch section 125. Meanwhile, since the fluid ejection section 101 is in an electrically non-drivable state, ejection of a pulsating flow from the fluid ejection section 101 due to an operation error by the operator is restrained. Also, the operator can learn that only the current application section 102 is in an electrically drivable state, since only the report section 45 in the second sealing state switching operation section 40A emits light.

As described above, according to the combination surgical device 100 of the second embodiment, a leakage of gas from inside the abdominal cavity via the handpiece 103 during a surgical operation is restrained by the first and second sealing sections 131, 132. Also, during a surgical operation, the fluid ejection section 101 and the current application section 102 can be replaced easily in the state where sealability of the abdominal cavity is secured. Moreover, the fluid ejection section 101 or the current application section 102 can be driven in the state where sealability in the handpiece 103 is enhanced. Also, driving of the fluid ejection section 101 or the current application section 102 in an unused state due to an operation error is restrained.

### C. Modifications

### C1. Modification 1

The laparoscopic surgical apparatus of the first embodiment is configured as the fluid ejection device 10 having the fluid ejection tube 30 and the ejection drive section 33 accommodated in the casing section 21 of the handpiece 20. However, the laparoscopic surgical apparatus of the first embodiment may not be configured as the fluid ejection device 10. The laparoscopic surgical apparatus of the first embodiment may be configured, for example, as an electric surgical knife. Specifically, instead of the fluid ejection tube 30 and the ejection drive section 33, the forward end surgical knife section 121 and the cable section 122 of the current application section 102 described in the second embodiment may be accommodated in the casing section 21 of the handpiece 20.

### C2. Modification 2

The combination surgical device 100 of the second embodiment has the fluid ejection section 101 as a first surgical instrument section, and the current application section 102 as a second surgical instrument section. However, in the combination surgical device 100 of the second embodiment, another surgical instrument section may be combined instead of the fluid ejection section 101 or the current application section 102. Alternatively, in the combination surgical device 100 of the second embodiment, another surgical instrument section may be combined in addition to the fluid ejection section 101 and the current application section 102. For example, in the combination surgical device 100, a camera having an image pickup element mounted at the forward end of a cable may be combined as a surgical instrument. Also, in the combination surgical device 100, a bipolar electric surgical knife may be combined.

### C3. Modification 3

The fluid ejection device 10 of the first embodiment is not in an electrically drivable state when the first sealing state is established. However, the fluid ejection device 10 may be in an electrically drivable state when the first sealing state is established. It is desirable that the fluid ejection device 10 is in an electrically drivable state when sealability in the handpiece 20 is secured. It is more desirable that the fluid ejection device 10 is in an electrically drivable state when the second sealing state is established. In the combination surgical device 100 of the second embodiment, the fluid ejection section 101 is not in an electrically drivable state when the first sealing section 131 is in the first sealing state, and the current application section 102 is not in an electrically drivable state when the second sealing section 132 is in the first sealing state. However, the fluid ejection section 101 may be in an electrically drivable state when the first sealing section 131 is in the first sealing state, and the current application section 102 may be in an electrically drivable state when the second sealing section 132 is in the first sealing state. It is desirable that the fluid ejection section 101 and the current application section 102 are electrically drivable when sealability is secured in the first and second sealing sections 131, 132. It is more desirable that the fluid ejection section 101 and the current application section 102 are in an electrically drivable state when the corresponding sealing sections 131, 132 are in the second sealing state.

By the way, in the first embodiment and the second embodiment, the “electrically drivable state” of the fluid ejection device 10 or the fluid ejection section 101 means the state where a fluid can be ejected from the nozzle 31, with an electric drive force. In the second embodiment, the “electrically drivable state” of the current application section 102 means the state where a high-frequency current can be outputted from the forward end of the forward end surgical knife section 121, with an electric drive force. In this way, in this description, the “electrically drivable state” of a surgical instrument means the state where the original function of the surgical instrument can be performed with an electric drive force, and this does not include the state where other functions than the original function of the surgical instrument can be performed with an electric drive force. Therefore, in the respective embodiments, the fluid ejection device 10, the fluid ejection section 101 and the current application section 102 may be in another state than the electrically drivable state when the first sealing state is established. For example, functions such as preheating and cleaning of component devices may be drivable with an electric drive force.

### C4. Modification 4

In the combination surgical device 100 of the second embodiment, the function capable of detecting that the first and second sealing sections 131, 132 are in the second sealing state is realized by the cooperation operation of the first and second electrode sections 41a, 41b, the contact detection unit 65 and the drive switch section 125. However, the combination surgical device 100 of the second embodiment may have the function of detecting that at least one of the first and second sealing sections 131, 132 is neither in the first sealing state nor in the second sealing state, with no sealability established in the handpiece 103. Specifically, the combination surgical device 100 of the second embodiment may have an electrode section or sensor section that can detect that the first and second sealing state switching operation sections 40, 40A are at the position on the furthest rear end side in the slidable range. In this configuration, if it is detected that no sealing state is established in one of the first and second sealing sections 131, 132, the control units 60, 140 of the fluid ejection section 101 and the current application section 102 may cooperate with each other in such a way that neither of the fluid ejection section 101 and the current application section 102 is in an electrically drivable state.

### C5. Modification 5

In the fluid ejection device 10 of the first embodiment, the nozzle 31 is accommodated in the casing section 21 when the first sealing state is established. However, in the fluid ejection device 10, the nozzle 31 may protrude from the casing section 21 when the first sealing state is established. In the combination surgical device 100 of the second embodiment, the nozzle 31 of the fluid ejection section 101 is accommodated in the exterior section 110 when the first sealing section 131 is in the first sealing state, and the forward end surgical knife section 121 of the current application section 102 is accommodated in the exterior section 110 when the second sealing section 132 is in the first sealing state. However, in the combination surgical device 100, the nozzle 31 of the fluid ejection section 101 may protrude from the exterior section 110 when the first sealing section 131 is in the first sealing state. Also, the forward end surgical knife section 121 of the current application section 102 may protrude from the exterior section 110 when the second sealing section 132 is in the first sealing state.

### C6. Modification 6

In the respective embodiments, the sealing state switching operation sections 40, 40A are configured as sliding operation sections capable of sliding in the extending direction of the handpieces 20, 103. However, the operation section for switching the sealing state in the handpieces 20, 103 may not be configured as a sliding operation section. The sealing state switching operation sections 40, 40A may be configured, for example, to move the sliding sealing section by rotating in the circumferential direction of the handpieces 20, 103. Also, the sealing state switching operation sections 40, 40A may be configured to push and move the sliding sealing section 37, using a lever-type operation section. Moreover, the sealing state switching operation sections 40, 40A may be configured as a button-type operation section and configured in such a way that a drive section moves the sliding sealing section 37 when the operator presses the button.

### C7. Modification 7

In the fluid ejection device 10 and the combination surgical device 100 of the respective embodiments, the inside of the handpieces 20, 103 is sealed by the fixed sealing section 36, 36A and the sliding sealing section 37. However, in the fluid ejection device 10 and the combination surgical device 100, the inside of the handpieces 20, 103 may be sealed by a sealing section having another configuration than the fixed sealing section 36, 36A and the sliding sealing section 37. For example, the inside of the handpieces 20, 103 may be sealed by a single annular sealing section mounted on the outer periphery of the fluid ejection tube 30 and the cable section 122.

### C8. Modification 8

In the respective embodiments, the sealing state in the handpieces 20, 103 is switched by changing the contact area between the fixed sealing section 36, 36A and the sliding sealing section 37. However, the sealing state in the handpieces 20, 103 may be switched by other methods. For example, an external force may be applied to a sealing section surrounding the outer periphery of the fluid ejection tube 30 and the cable section 122, thereby increasing the contactability of the sealing section to the fluid ejection tube 30 and thus switching the sealing state. Alternatively, the fluid ejection tube 30 and the cable section 122 may be configured to change in diameter in the extending direction, and the fluid ejection tube 30 and the cable section 122 may be moved in the extending direction with respect to the sealing section, thereby changing the contactability of the sealing section.

### C9. Modification 9

In the fluid ejection device 10 and the combination surgical device 100 of the respective embodiments, the sealing states of the fixed sealing section 36, 36A and the sliding sealing section 37 and the control of the electric driving of the surgical instrument section are interlocked with each other via the contact state of the first and second electrode sections 41a, 41b. However, in the fluid ejection device 10 and the combination surgical device 100, the sealing states of the fixed sealing section 36, 36A and the sliding sealing section 37 and the control of the electric driving of the surgical instrument section may be interlocked with each other via other components than the first and second electrode sections 41a, 41b. For example, in the fluid ejection device 10 and the combination surgical device 100, the control units 60, 140 may detect whether it is the second sealing state or not, on the basis of change in the contact resistance between the fixed sealing section 36, 36A and the sliding sealing section 37, and control the electric driving of the ejection drive section 33, the fluid ejection section 101 and the current application section 102 on the basis of the result of the detection.

### C10. Modification 10

In the respective embodiments, the fluid ejection device 10 and the combination surgical device 100 are provided with the report section 45 for reporting to the operator that it is the second sealing state where the surgical instrument section is electrically drivable. However, in the fluid ejection device 10 or the combination surgical device 100, the report section 45 may be omitted. Also, the report section 45 may report to the operator that it is the second sealing state where the surgical instrument is electrically drivable, by other methods than emitting light (for example, displaying a mark or the like). The fluid ejection device 10 and the combination surgical device 100 may be provided with a report section that reports that it is the first sealing state, in addition to the report section 45 reporting that it is the second sealing state. Alternatively, the report section 45 may change the color of emitted light so as to report the first and second sealing states differently.

### C11. Modification 11

In the second embodiment, the first and second sealing state switching operation sections 40, 40A are operable independently of each other. However, the operations of the first and second sealing state switching operation sections 40, 40A may be interlocked with each other. Specifically, when first sealing state switching operation section 40 is at the position of the second sealing state, the second sealing state switching operation section 40A may be locked at the position of the first sealing state, whereas when the second sealing state switching operation section 40A is at the position of the second sealing state, the first sealing state switching operation section 40 may be locked at the position of the first sealing state. Also, when first sealing state switching operation section 40 moves to the position of the first sealing state from the position on the rear end side, the second sealing state switching operation section 40A, too, may move to the position of the first sealing state from the position on the rear end side.

The invention is not limited to the above embodiments, examples and modifications and can be realized with various configurations without departing from the scope of the invention. For example, the technical features included in the embodiments, examples and modifications corresponding to the technical features included in the configurations described in the summary section can be properly replaced or combined in order to solve a part or all of the foregoing problems or in order to achieve a part or all of the advantageous effects described above. Also, the technical features can be properly deleted unless described as essential in the description.

What is claimed is:

1. A laparoscopic surgical apparatus comprising:
a surgical instrument section that has an extension site having at least a forward end part thereof inserted into an abdominal cavity, and is driven by electric power supplied from outside;
a cylindrical accommodation section that accommodates the extension site of the surgical instrument section in such a way that the extension site is movable in an extending direction; and
an operation section for causing the extension site of the surgical instrument section to move in the extending direction from outside the accommodation section;
wherein a sealing section for sealing an inside of the abdominal cavity off from outside is arranged between the surgical instrument section and the accommodation section,
the sealing section is capable of shifting from a first sealing state with a first airtightness to a second sealing state with a higher airtightness than in the first sealing state, through the movement of the extension site by the operation section, and
the surgical instrument section is in an electrically drivable state when the second sealing state is established.

2. The laparoscopic surgical apparatus according to claim 1, wherein the surgical instrument section is in another state than the electrically drivable state when the first sealing state is established.

3. The laparoscopic surgical apparatus according to claim 1, wherein the sealing section is in the first sealing state when the forward end part of the extension site is accommodated inside the accommodation section, and the sealing section shifts to the second sealing state when the forward end part of the extension site extends from the accommodation section through the movement of the extension site by the operation section.

4. The laparoscopic surgical apparatus according to claim 1, wherein the surgical instrument section includes first and second surgical instrument sections, each having an extension site extending in parallel within the accommodation section,
the operation section includes first and second operation sections for operating the respective extension sites of the first and second surgical instrument sections,
the sealing section includes first and second sealing sections provided between the respective extension sites of the first and second surgical instrument sections and the accommodation section,
the first surgical instrument section is in an electrically drivable state when the first sealing section is in the second sealing state, and
the second surgical instrument section is in an electrically drivable state when the second sealing section is in the second sealing state.

5. The laparoscopic surgical apparatus according to claim 1, wherein the operation section has:
a slider section that is slidable outside the accommodation section; and
a cylindrical holding section that airtightly holds an outer periphery of the surgical instrument section inside the accommodation section and is connected to the slider section,
the extension site of the surgical instrument section moves within the accommodation section along with the holding section when the slider section is operated to slide,
the sealing section is arranged to annularly surround an outer periphery of the extension site, and
the first sealing state of the sealing section is a state where an inner peripheral part of the sealing section and an outer peripheral part of the holding section contact each other airtightly, and the second sealing state is a state where the holding section is situated deeper into the sealing section than in the first sealing state.

6. The laparoscopic surgical apparatus according to claim 5, further comprising:
a drive control unit that controls electric driving of the surgical instrument section;
a first electrode section that moves, interlocked with the slider section; and
a second electrode section fixed to the accommodation section,
wherein the first and second electrode sections contact each other when the sealing section moves to a position where the second sealing state is established, through the sliding of the slider section, and
the drive control unit performs control in such a way that electric power from outside can be supplied to the surgical instrument section when the first and second electrode sections contact each other.

7. The laparoscopic surgical apparatus according to claim 1, further comprising a report section that reports that the surgical instrument section is in an electrically drivable state.

8. The laparoscopic surgical apparatus according to claim 1, wherein the surgical instrument section includes a fluid ejection section that ejects a fluid to an operative field inside the abdominal cavity from the forward end part.
